(19) United States
(12) Patent Application Publication
(10) Pub. No.: US 2015/0133748 A1
(43) Pub. Date: May 14, 2015

(54) INTERACTIVE SYSTEM FOR MONITORING AND ASSISTING THE PHYSICAL ACTIVITY OF A USER WITHIN A GYM ENVIRONMENT

(71) Applicant: Extronics Pty Ltd, Brisbane (AU)

(72) Inventors: Adrian Anthony Edmonds, Brisbane (AU); Vladimir Rapaic, Brisbane (AU); Pavle Krkeljas, Brisbane (AU)

(21) Appl. No.: 14/514,233

(22) Filed: Oct. 14, 2014

(30) Foreign Application Priority Data
Oct. 14, 2013 (AU) 2013903941

(51) Int. Cl. A61B 5/22; A61B 5/0205; A61B 5/11; A61B 5/00

(52) U.S. Cl. CPC A61B 5/222; A61B 5/6806; A61B 5/002; A61B 5/6898; A61B 5/0022; A61B 5/0205; A61B 5/1118; A61B 5/1121; A61B 5/7475; A61B 5/742
USPC 600/301; 600/300; 600/508; 600/485; 600/595

(57) ABSTRACT

Apparatus and systems for monitoring and assisting with the physical activity of a user within a gym environment. In one embodiment, the present disclosure relates to providing a system for monitoring the user and providing the user a manner in which to interact with the various components of the gym environment so as to provide a favorable experience and motivational feedback. The present disclosure also provides systems and devices that can be added by, for example, a gymnasium to existing infrastructure and therefore provide a particular gym with a technological edge against its competitors.

### PRIORITY

This application claims the benefit of priority to co-owned Australian Provisional Patent Application No. 2013903941 of the same title filed Oct. 14, 2013, the contents of which are incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

1. Technological Field

The present disclosure relates to a system for monitoring and assisting the physical activity of a user within a gym environment. More particularly, the present disclosure relates to providing a system for monitoring the user and providing the user a manner in which to interact with the various components of the gym environment so as to provide a favorable experience and motivational feedback.

2. Description of Related Technology

The monitoring of physical activity of a user employing devices and systems within the fitness sector is well known. These devices and systems are commonly used to measure one or more parameters of the user activity and or physiological state during an exercise routine so as to provide feedback and reports to assist the user in reaching a predetermined fitness goal. Specifically, systems within the prior art generally comprise a portable smart phone device or the like that is placed into the pocket of a user or strapped to an item of clothing, such that it is held within close proximity to the user. Using this close proximity to the user and the gym equipment the device in real-time captures a number of signals proportional to the physical activity performed.

For example, Great Britain patent publication no GB 2454705 discloses a system that proposes to monitor the physical activity of a user using a wearable device having one or more accelerometers. The wearable device uses signals measured by the accelerometers to derive parameters relating to the intensity of the exercise, which are then stored locally and remotely of data storage devices so as to give feedback and reports on the exercise activity and progress.

United States Patent Publication No.: 2012/0094649 primarily discloses a system for monitoring the physical activity of a user, which uses a mobile phone or smart phone device (14) in conjunction with a portable medium (3) designed to be worn by the user, the portable medium is adapted with sensors to detect a signal relating to movement of the user. A remote server wirelessly receives data from the portable medium which is also in communication with the mobile phone device, whereby the server after processing the data transmits real-time motivational indications to the user via the display unit of the mobile phone device.

Yet another example of an activity monitoring system is that disclosed by United States Patent Publication No.: 2012/0259649. The systems comprise a communication interface, a database to store treatment information and sensor data recorded from sensors worn by the user during exercise or movements. The monitoring system uses the recorded data to provide motivational indications to the user, which are sent from the server and are merely a linked to the level of physical movement (i.e. speed of running as a function of time).

While the above systems do provide manners in which to monitor the physical activity within a gym environment and use such information in some embodiments to motivate the user, the systems are rather user based and personal. In other words, the systems of the prior art rely on the user to supply certain components of the system, such as a smart phone or the like that ultimately provides the exercise performance statistics to the user. This tends to take control away from the gym and the professionals of the gym (i.e. personal trainers) who have the ability to monitor the user and ensure they are meeting their predetermined fitness goal.

Moreover, systems of the prior art tend to only be directed to providing information with respect to motion and/or physiological parameters in a convenient way and do not provide a complete picture in which to analyse the progress of a person during training. Prior art systems do not provide sufficient information in which to gauge whether or not a particular exercise has been performed correctly and thus does not place any accountability on the user during the training. Additionally, it would be advantageous to provide a monitoring system that allows personalization to a specific user so as to provide the user with a tailor made solution to ensure the desired results and experience is achieved.

As such, there is a long felt need in the art for novel and improved interactive system for monitoring and assisting the physical activity of a user within a gym environment. There is also a need to provide systems and devices that can be simply added by a gymnasium to existing infrastructure and to provide a particular gym with a technological edge against its competitors.

### SUMMARY

In a first aspect, an interactive system for monitoring the physical activity of a user within a gym environment is disclosed. In one embodiment, the interactive system includes a local server having one or more databases storing personal data comprising at least an exercise history data and a pre-set exercise regime data for the user; a smart device associated with a first gym equipment, the smart device having a display unit, a wireless communication apparatus and a processor, the smart device configured to permit an authenticated user to access and display their personal data on the display unit downloaded from the local server; a wearable sensing device provided and assigned by the gym environment having a plurality of sensors configured to measure and record one or more exercise parameters of the user during use of the first gym equipment, the wearable sensing device further comprising a sensing device wireless communication apparatus; the smart device being further configured to receive and process the recorded one or more exercise parameters from the wearable sensing device to thereby display and record real-time exercise statistics of the user during use of the first gym equipment on the display unit; where the smart device is further configured to wirelessly transmit the exercise statistics to the local server to compile and update the exercise history data associated with the user.

In one variant, the system further includes a remote database or a cloud storage configured to back-up the personal data via a wide area network (WAN).

In another variant, the user is permitted to access the personal data stored on the remote database or the cloud storage via a web portal following authentication therewith.

In yet another variant, the wearable sensing device is assigned a fixed IP address within a local area network (LAN) within the gym environment, the fixed IP address being assigned to the user upon logging into the system in order to assist the local server in recognizing the user during a workout period.

In yet another variant, the smart device is in communication with the wearable sensing device whereby the wearable sensing device is configured to permit the user to alter one or more exercise settings on the smart device.

In yet another variant, the one or more exercise settings comprises an amount of weight being lifted by the user during the exercise and/or a number of repetitions and sets being performed.

In yet another variant, the system further comprises a first near field communication (NIT) device associated with the smart device.

In yet another variant, the wearable sensing device further includes a second near field communication (NFC) device.

In yet another variant, bringing the first and second near field communication (NFC) devices in proximity to each other permits the user to be authenticated with the smart device.

In yet another variant, the real-time exercise statistics comprises one or more of intensity, rate of repetitions during the exercise, number of repetitions carried out during the exercise, heart-rate, and blood pressure.

In yet another variant, the wearable sensing device further comprises one or more magnetic sensors adapted to detect proximity with the first gym equipment.

In yet another variant, the wearable sensing device is configured to be attached to an item of clothing worn by the user.

In yet another variant, the wearable sensing device includes a wrist band configured to be secured around the wrist of the user.

In yet another variant, the local server transmits real-time information to the smart device to provide motivational indications to the user based on the recorded and processed one or more exercise parameters that are constantly compared to a pre-determined goal.

In yet another variant, the smart device is selected from the group consisting of a tablet device, a PDA device and a computer.

In a second aspect, a wearable sensing device is disclosed. In one embodiment, the wearable sensing device includes a processor; a power supply; a motion sensor; a storage apparatus; and a wireless communication apparatus. The wearable sensing device is further configured to communicate with an interactive system for monitoring the physical activity of a user within a gym environment.

In one variant, the wearable sensing device includes at least three gravitational measurements; at least three rotational measurements; and at least three magnetic measurements.

In another variant, the motion sensor includes one or more accelerometers.

In yet another variant, the device further includes an input interface apparatus.

In yet another variant, the device further includes a status indicator light, the status indicator light configured to provide to a user a visual indication of their performance during a particular exercise.

### BRIEF DESCRIPTION OF THE DRAWINGS

The features, objectives, and advantages of the disclosure will become more apparent from the detailed description set forth below when taken in conjunction with the drawings, wherein:

FIG. 1 illustrates an interactive system for monitoring the physical activity of a user within a gym according to a preferred embodiment;

FIGS. 2A and 2B illustrate a wearable sensing device of the present disclosure adapted to be worn with a glove of a user;

FIGS. 3A and 3B illustrate a wearable sensing device of the present disclosure adapted to be worn with a shoe of a user;

FIG. 4 illustrates a schematic of various components of the wearable sensing device according to a preferred embodiment of the present disclosure; and

FIG. 5 illustrates the association between a wearable sensing device and a smart device according to an embodiment of the present disclosure, thus illustrating how various parameters on the smart device can be manipulated via the input interface on the sensing device.

All Figures disclosed herein are © Copyright 2013 Extronics Pty Ltd. All rights reserved.

### DETAILED DESCRIPTION

Reference is now made to the drawings wherein like numerals refer to like parts throughout.

### Detailed Description of Exemplary Embodiments

Detailed descriptions of the various embodiments and variants of the apparatus and methods of the disclosure are now provided. While primarily discussed in the context of a gym setting, the various apparatus and methodologies discussed herein are not so limited. In fact, many of the apparatus and methodologies described herein are useful in any number of other fields including, without limitation, horse racing, dog racing, swimming and athletics that can benefit from the measured activity parameter methodologies and apparatus described herein.

Interactive System for Monitoring Physical Activity—

In a first embodiment of the present disclosure, an interactive system for monitoring the physical activity of a user within a gym is provided. More directly, the system of the present disclosure relates to systems for measuring and recording one or more exercise parameters during an exercise. In this instance, the term “exercise parameters” is intended to refer generally to data that is received and processed by the system of the present disclosure during an exercise, and includes but is not limited to, movement of the user, one or more physiological parameters (e.g. heart rate, blood pressure, and the like), repetitions of an exercise and intensity. The data measured, processed, and recorded can collectively be referred to as “exercise data”.

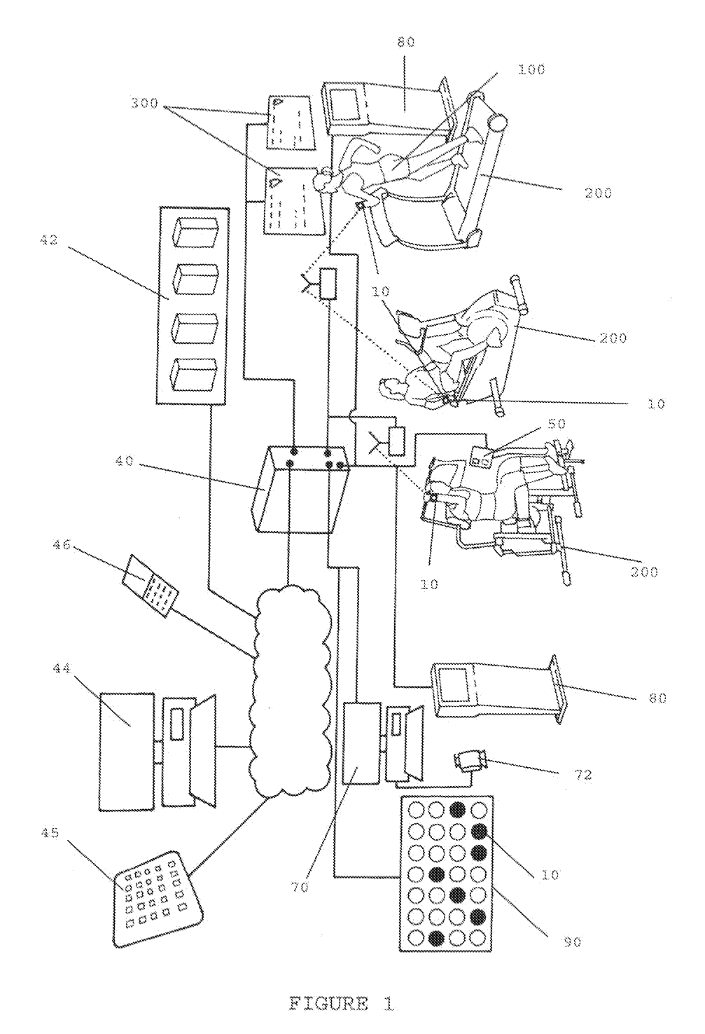

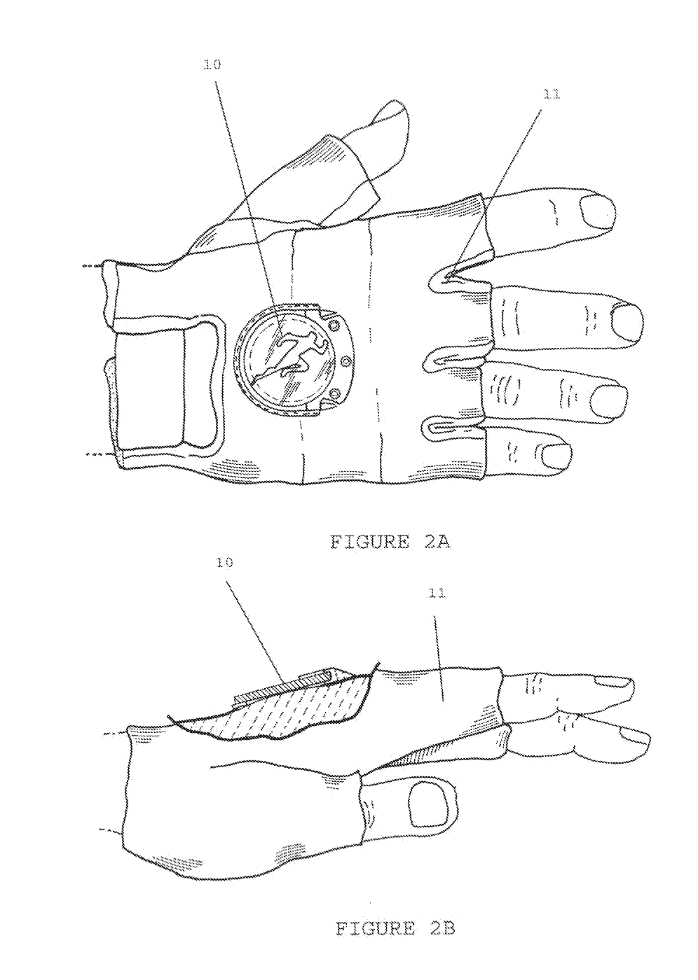

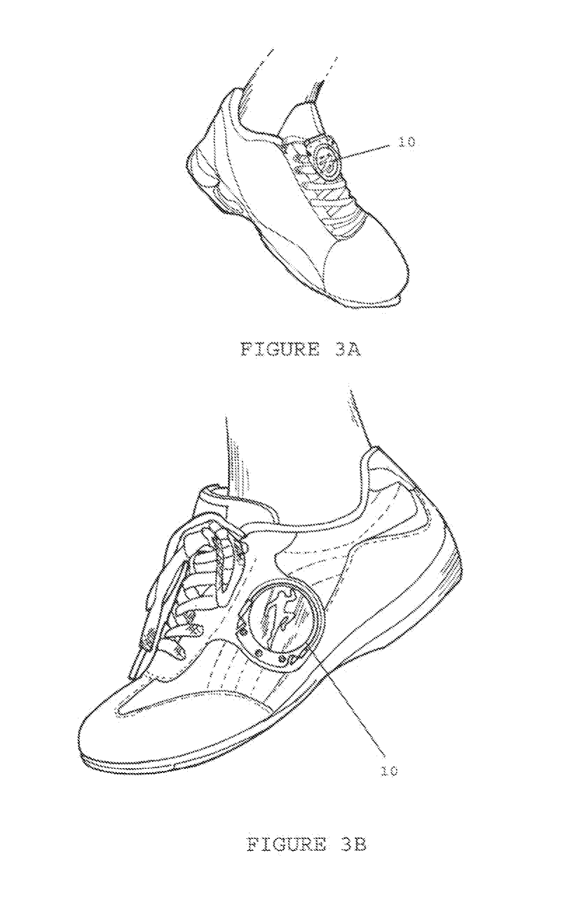

Referring now to FIG. 1, an interactive system for monitoring the physical activity of a plurality of users is shown and described in detail. A wearable movement and activity sensing device 10 is provided and is adapted to be worn by a user. To highlight the features of the wearable movement and activity sensing device in further detail, reference will be now made to FIGS. 2-4, which show this activity sensing device in greater detail. For example, as can be seen in FIGS. 2A-2B and 3A-3B, the wearable sensing device 10 is configured to, for example, be a clip-on device that may be attached to a user's clothing (e.g. a glove 11 or shoe 12 worn by the user shown in FIGS. 2 and 3, respectively). It will be appreciated that any suitable attachment means can be used to secure the sensing device to the item of clothing. These attachment means may be fixed (e.g., sewn on or otherwise incorporated into the underlying article of clothing) or removable (e.g., via the use of snaps. Velcro™, zippers, etc.).

It will also be appreciated that the form of the wearable sensing device is not overly limited, and those skilled in the art would comprehend other forms to fall within the scope of the present disclosure without departing from the spirit thereof. For example, it is envisaged that the sensing device may in fact be in the form of a watch or band to be worn by the user around their wrist, ankle and/or waist.

As will be discussed in further detail in the following paragraphs, the wearable sensing device 10 is adapted to measure one or more exercise parameters relating to a user's interaction with their environment (e.g., a piece of gym equipment during a particular exercise routine being carried out). The term “interaction” in this instance is intended to define how the wearable sensing device moves in a three dimensional space during a gym exercise or routine carried out by a user. This is achieved by, for example, the provision of one or more motion sensors.

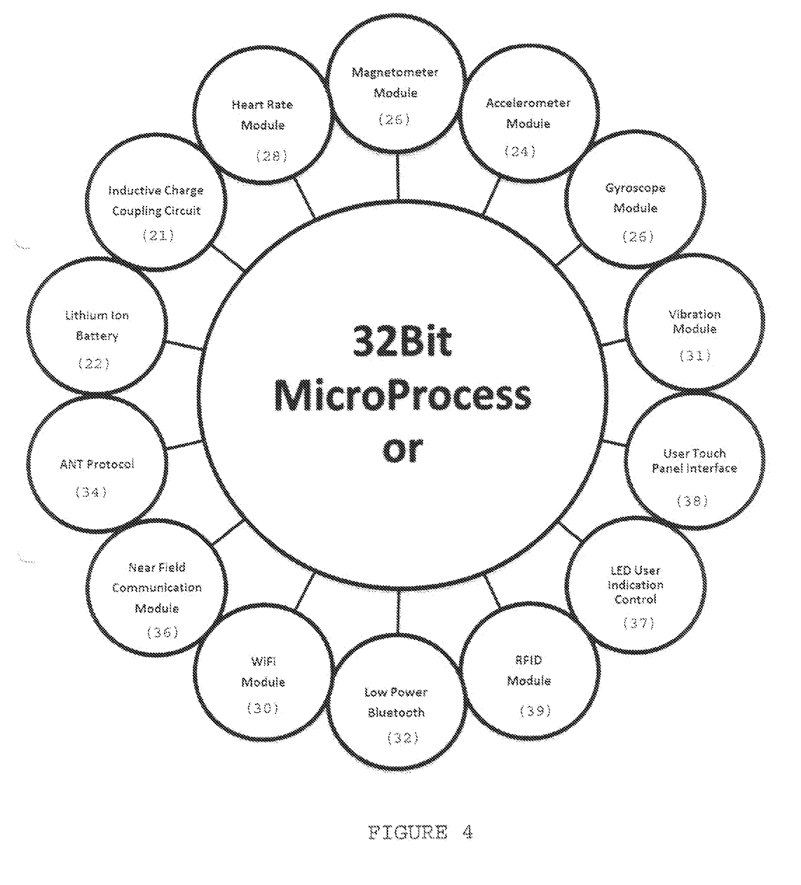

Referring now to FIG. 4, a diagrammatic representation of the components of the sensing device 10 is shown and described in detail. In its broadest sense, the sensing device 10 comprises one or more integrated electronic modules housed within an outer case that is hermetically sealed. The sensing device includes a processor 20 (e.g., a 32-bit processor), which functions to pre-process the one or more exercise parameters prior to streaming the data to the server via the Wi-Fi module 30. The sensing device also includes a power supply 22, which is, in an exemplary implementation, a rechargeable power supply including but not limited to a lithium-ion based battery. The sensing device can in some or all embodiments also include a battery status indicator 37 to indicate the charged state of the battery, and in a particularly preferred embodiment, is charged by the use of a non-contact inductive coupling mechanism 21.

In one embodiment, the battery status indicator 37 is also adapted to provide a visual indication to the user as to the status of the exercise (e.g., when the device is in use). For example, when an exercise has been performed correctly a green light or other suitable color may be displayed. The manner in which the status indicator is used is not intended to be overly limiting, and hence can be used to visually display any information to the user simply and quickly during an exercise. It is also envisaged that the vibration module 31, which will be explained in further detail subsequently below with respect to a sensing function, may also function to provide vibration when the battery charge is low.

The wearable movement and activity sensing device 10 of the present disclosure also comprises a motion sensor module, which in exemplary embodiments, comprises accelerometers 24 that are adapted for sensing acceleration along three orthogonal axes (i.e., commonly referred to as the x, y and z axes/planes). The motion sensor module may further comprise additional accelerometers and/or, for example, micro-electromechanical systems (“MEMS”) based gyroscopes 25 where necessary in order to measure angular motion and rotational motion about the three orthogonal axes x, y and z. However, it would be understood by those skilled in the art that the number and configurations of the accelerometers used in the sensing device can be readily adapted in view of the motion intended to be measured.

In order to provide additional information and measurements relating to a user's activity, the sensing device 10 in some or all embodiments comprises one or more additional measurement devices such as magnetic measurement devices 26. The magnetic measurement devices are included in order to interact with magnetic members that may be associated at a location with/on a particular piece of gym equipment 200. The magnetic measurement devices 26 measures and records when they are moved in close proximity to the magnetic member of the gym equipment, and thus can be useful to detect the user moving into and out of close proximity of a particular exercise machine. For example, this proximity reading is contemplated to be used to record the beginning and ending of a particular exercise. It will be appreciated that the distance meant by close proximity in this instance is not meant to be overly limiting and can be set accordingly by those skilled in the art.

In one exemplary embodiment, the sensing device worn by a user measures with nine (9) degrees of freedom which includes, three (3) gravitational measurements, three (3) rotational measurements and three (3) magnetic measurements. This ensures sufficient information can be provided and processed by the sensing device to give an accurate reading of the user's interaction within the gym environment. Preferably, the sensing device 10 also includes a vibration module 31 that is used to detect and record vibration, which can correlate to physical activity and use of the device. The vibration module is also configured to provide a vibration in response to a particular programmed event, such as and not limited to, when the user is about to or has completed their set or selected time during a workout.

It is also envisaged that the sensing device comprises one or more physiological parameter measurement devices. The one or more physiological parameter measurement devices measures and records the physiological state of the user while carrying out the exercise and also during the recovery period. This includes measurements relating to, but not limited to, parameters selected from the group consisting of heart-rate, skin temperature, perspiration, blood oxygen levels, glycogen depletion and a combination thereof. As shown in FIG. 4, a heart rate module (28) is also included.

The wearable movement and activity sensing device 10 also includes a processor and a memory device (not shown). The memory device includes, for example, random access memory (“RAM”) or another suitable type storage medium routinely adapted by a person skilled in the art. The purpose of this memory device is to collect and record all relevant exercise data and parameters provided from the motion sensors and processed by the processor, which can be accessed at a later time and date to permit analysis thereof In particularly preferred embodiments, the memory device is configured to only temporarily store the data until a time that it is unloaded and replicated through synchronization with a local database and/or a cloud storage means, which permits a person to access the workout data either locally within the gym or remotely through a web portal or a smart device application.

In this regard, in order to exchange recorded data with databases and storage mediums included within the system, the wearable sensing device 10 has at least one data transfer interface. The data transfer interface in one embodiment includes an interface to allow a direct or wired connection with a computer or like device within the network. This wired connection comprises without limitation a universal serial bus device (USB) or micro-USB technology or the like. In other embodiments, the data transfer interface would include a wireless communication means which is IEEE 801.11 compliant 30, Bluetooth® compliant 32 or ANT+ technology 34. While it should be appreciated that only one type of data interface would achieve the purposes of the data transfer, those skilled in the art would appreciate that both a physical connection and wireless communication means could be built in to the wearable sensing device where appropriate. The sensing device may also in certain embodiments, as shown in FIG. 4, a RFID module 39.

The Wi-Fi capabilities of the sensing device 10 are also important to permit the device to wirelessly communicate with other elements of the interactive fitness monitoring system of the present disclosure. In preferred embodiments, when in use and as shown best in FIG. 1, the sensing device 10 is in wireless communication (shown as dotted lines) with wireless access points 31. The wireless network architecture enables wireless communication between various elements of the system, such as the smart device 50 that is associated with the piece of gym equipment 200 that the user is currently using and logged into, which in turn permits the sensing device and the smart device to process and display real-time exercise data.

Once again referring to FIG. 4, the sensing device 10, in some embodiments, also includes a near field communication (“NFC”) device 36 that is used to allow easy and quick communication with other components in close proximity, such as linking the sensing device with the smart device 50 included in the system of the present disclosure as discussed previously and/or other future devices that may be incurred into the system.

In particular preferred embodiments, the wearable movement and activity sensing device 10 comprises an interface device 38, so as to provide the user touch screen functionality. This interface provides the user with a means by which to manipulate settings and select preferences for a particular exercise being performed, which are displayed on the smart device. By way of a non-limiting example, due to the fact that use the sensing device 10 is linked to the smart device via the network, the interface provides the user 100 with a wireless means in which to input changes to the exercise routine, such as the weight being lifted, the number of sets and repetitions being performed. While the interface device 38 of the sensing device 10 is provided, the user is also provided with a means in which to input these changes directly to the smart device when desired.

As also shown in FIG. 1 and denoted by numeral 40, the system of the present disclosure comprises a local database server, which is adapted to store personal information of the user as well as the exercise data recorded over a period of time and history. Only an authenticated user has the ability to access the stored data via a local or remote portal. In some or all embodiments and as designated by numeral 42, the system of the present disclosure further comprises a remote server database or cloud server database. Without limitation, the one or more databases of the present disclosure comprise biometric information about the user 100, such as, for example, age, weight, sex and details about the lifestyle of the user 100, as well as recorded exercise data and a predetermined fitness goal.

In preferred aspects of the present disclosure, the user can connect up to the remote server database 42 from a computer 44, tablet 45 and/or smart phone 46 using a web portal via an internet connection, so as to receive and review the exercise data relating to the user. The user 100 can also allow a third party, such as a fitness instructor, access to their file via a similar web portal to allow review against a pre-determined fitness regime. Without limitation, the user accesses a web portal hosted on the remote server database and logs in to their personal record, which as explained below is compiled data previously recorded by the wearable sensing device 10 of the present disclosure.

By accessing the historical exercise data via the web portal, the user or authorized third party can manipulate the data to provide a plurality of exercise statistics or other processed information, such as a graphical representation of weight loss, intensity during exercise and any other measured parameters. Using this information the user and any authorized third party viewing the same can easily determine the user's progress.

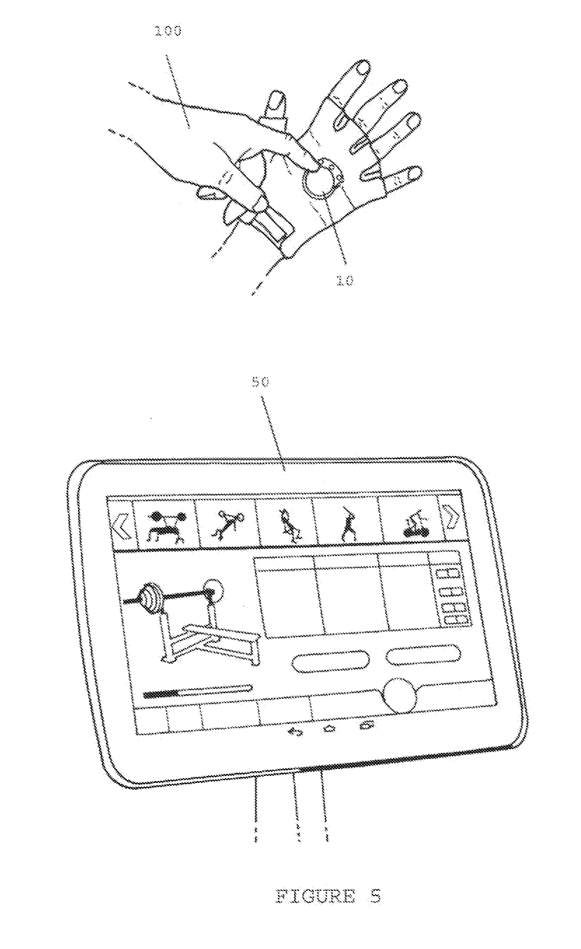

As is shown, each piece of equipment 200 within the gym is associated with a smart device 50, which can include but not be limited to a tablet computer. In a broad sense, the smart device comprises a display unit, a wireless communication means and a central processing unit (“CPU”). The smart device is adapted to display the one or more exercise parameters in real-time, which as will be discussed in further detail throughout the detailed description, is collected and streamed from the wearable sensing device. This on screen information provided by the smart device is achieved by a program or apples installed and running thereon, as is shown best in FIG. 5B.

In effect, the real-time information displayed on the smart device 50 is intended to provide motivational indications to the user 100 and permit the user to compare their progress and results against a pre-determined fitness goal, as well as reminding the user 100 which exercises are to be performed at a specific time and date. It also provides information as to the progress of the exercise, such as number of repetitions and sets remaining for a particular routine. In some embodiments, the smart device may also be programmed to download the user's pre-set exercise program to prompt the user what exercise is next after completion of their exercise that is in progress. For instance, for a particular day of the week, the user may have a program to concentrate on one particular body area and thus the smart device 50 can download suggestions for a particular exercise from the server 40.

To facilitate comparison and review of the one or more exercise parameters measured during a particular exercise, the smart device 50 also comprises a storage or memory device. As will be discussed below with reference to use of the present disclosure, the exercise data is temporarily stored on the storage or memory device of the smart device 50 until a synchronization event takes place with the local database server 40. In a preferred embodiment of the present disclosure, this synchronization event is executed by the user logging out of the particular smart device 50 being used or a time-out event being triggered due to no exercise occurring with a pre-determined period of time.

In use of the system of the present disclosure, a user 100 at a particular gym is assigned with a unique identifier (username) and a password that enables the user to sign into and use the system on arrival to the gym for a workout, such as via an administrator computer 70. Once signed in and registered within the system, the user 100 is either automatically assigned a default exercise program for that particular day or alternatively a program for particular days and/or times can be selected by the user on advice from the personal trainer. It is envisaged that this could be done using a web portal or portal located with the local area network within the gym, via a number of kiosks 80 positioned in the gym.

When not in use and in particular preferred embodiments, it is envisaged that the wearable movement and activity sensing devices 10 are stored on a non-contacting inductive recharging panel 90 and constantly connected to the wireless network at all times and are assigned a fixed IP address while they have battery power. The sensing devices 10, as described previously, have a status indicator to indicate both its charged state and its connection status to the server of the system over the wireless network.

Once the user 100 enters the gym, the user swipes their membership card with a bar code reader (which is a normal occurrence) or the like after which they are assigned with or collect one or more wearable movement and activity sensing devices. Once collected the sensing device/s 10 are swiped across a near field communication reader 72, which permits the user to linked to the sensing device 10 and be identified with fixed IP address thereof during their workout period. While it is envisaged that a near field communication (“NFC”) reader, other ways of assigning the sensing device 10 to the user 100 are within the scope of the present disclosure without departing from the scope thereof, such as an auto-allocation procedure that initiates after the sensing device(s) are removed from the charging platform 90. In any event, the assigning step ensures that during a particular workout period while the user 100 is in the gym and has yet to sign out from the system, they can be identified in the system for that particular point in time by the fixed IP address assigned to the sensing device 10 within the network.

At this time it is also possible for further users (not shown) to be assigned a sensing device of their own, where the further user(s) may wish to follow the same exercise regime as the first user 100. This provides the option for a partner or friend to also move through the exercise regime and provide alternating support (also referred to as “spotting”) to each other. As each user would be wearing a sensing device 10, data is recorded whether or not a particular user is carrying out the particular exercise or providing support, and thus spotting activity can also be added to their exercise profile of the server. Each user may also provide feedback on each other during a linked workout, which can be advantageous from a review point of view to ensure a user reaches their fitness goal. While this is an advantageous point of the present disclosure, for ease of explanation, the further description will be provided as to a single user.

After being assigned a sensing device 10 for the particular workout period, the user is then permitted to make their way to an item of gym equipment 200 so as to carry out their first exercise. Prior to commencement of the exercise the user logs into the smart device that is associated with the piece of gym equipment. As described previously throughout the detailed description, the smart device 50 is preferably a tablet or computer device that has a display unit and a wireless communication means along with other computer circuitry. The smart device comprises a suitable operating system installed thereon and is running an executable program/software or applet that provides the user with an interface therewith to view and manipulate certain parameters relating to the exercise, as well as view their personal information and exercise history stored on a database within the network architecture.

Logging into the smart device 50 is achieved by a number of ways routinely understood by those skilled in the art. Preferably, this includes entering a chosen username and password selected by the user on signing up to the gym, or in light of the date that the sensing device assigns the user a fixed IP address during their exercise period a drop list of all active users for a given period of time within the gym could also enable the user to select their username there-from to log into the smart device. Once selected the user is required to enter their chosen password to ensure authentication is complete, or alternatively, this could be done by swiping their sensing device 10 across a NFC device associated and connected with the smart device 50. Once authenticated a notification is sent to the admin server computer 70 that is within the network architecture, which ensures that any data received from the user carrying out physical activity on that particular piece of gym equipment and recorded can be associated with the relevant smart device, user and sensing device.

Now that the user has been authenticated with the smart device 50 and assigned a wearable sensing device 10 for their time at the gym, they are now free to begin the exercise. As soon as the exercise begins, the sensing device 10 begins measuring, recording and streaming the one or more exercise parameters. In real time, the smart device 50 displays information relating to exercise statistics processed from data received from the sensing device. This allows information such as repetition counting, intensity, tempo and the like, along with physiological data (i.e. heart rate, blood pressure etc.) to be displayed to the user.

The above is repeated at the next exercise station or piece of equipment until the user completes their entire workout. The authentication step at the smart device 50 in preferred embodiments logs the user out of all other smart devices they may have previously been authenticated and logged into. As the user 100 is logged out of each smart device, the exercise data recorded during use of that piece of equipment will be replicated and stored on the local database 40, and where required, backed up further Onto a remote storage means such as a cloud database server 42. Once the data from the smart device 50 has been replicated on the one or more databases, all data relating to the users exercise is deleted therefrom for privacy.

On completion of their exercise for a particular day, the user 100 returns the one or more sensing devices 10 to the charging station 90 where a complete log out of the system occurs. This ensures that the sensing devices assigned to the user during the workout are no longer associated with the user after they leave the gym.

At any time, the user 100 can access their personal data and exercise history stored on the local database or cloud server either from a kiosk computer 80 installed at the gym or remotely using a web portal. The user can also provide a personal fitness instructor access to selected information to ensure proper training is being achieved so as to meet a predetermined fitness goal, as well as change a particular exercise program based on performance and progress.

In a preferred embodiment, during the exercise, the exercise statistics measured may also be accessed and streamed to a secure tablet assigned to the personal trainer within the gym. This may include sending alerts to the personal trainer that a particular exercise is not being performed correctly by the user. Additionally, the smart device 50 may also provide the user with an option to request personal trainer assistance directly by selecting the option on the touch screed of the smart device. Once this is selected an alert is sent to the personal trainer for a personal consult, who then makes their way to the place where the user is carrying out the exercise. While the personal trainer is observing the user 100 during the exercise, they are provided with the option by way of their handheld table to change the user's exercise regime (i.e. weight to be lifted, repetitions etc.) according to their performance. Alternatively, a meeting may be automatically set up by the personal trainer for the user when they are not immediately available for consultation.

The system of the present disclosure may also include a number of display units 300 around the gym (i.e. LCD televisions or the like), especially associated and viewable from cardio workout machines (i.e. elliptical, rowing machines, bicycles, and treadmills). For instance, it is envisaged that each of the treadmills would be connected in a group whereby the user's using them at a particular time can view their performance in comparison with other users. This gives the user 100 an option to have contests with other users within the gym and thus adds another element of motivation to the system. It is also envisaged that the winning user or best performer for a particular exercise or time at the gym may have the option to select the next song playing in the gym, thus giving an incentive to the users to outperform other users. This incentive means is envisaged not only to be associated with cardio workout machines and thus can be associated with other areas and pieces of gym equipment around the gymnasium.

While it is perceived that there are many advantages of the present disclosure over the prior art, without limitation, they include:
The present disclosure allows a user to keep track of training progress over any particular period in time, by storing pertinent information in a database accessible via locally at the gym or remotely via a web portal. The system provides a number of levels of security architecture to enable personal information to be stored in a secure manner and only accessed by personal given security clearance by the user.
By having all workouts and information recorded it allows the user to easily compare their progress against a pre-determined fitness goal, a controlled training environment can be provided. The pre-determined fitness goal can be set according to the client during a consultation with a personal trainer or individual set, and thus milestones in the training regime allow the user to be kept on track by reminders and recommendations that are set by the system.
As the system records physical parameters in real time, such as repetitions, intensity and other motion parameters, poorly performed exercises can be flagged by the system to ensure follow up with a personal trainer. This ensures exercises are done properly and thus assists the pre-determined goal being met.
As the system of the present disclosure has an information station for each piece of gym equipment that communicates in real time with the wearable device, during the exercise real-time information can be displayed on the information station to ensure the exercise is done correctly. This includes the ability to display a live interactive tutorial to understand the correct usage of the gym equipment.
As each component of the system of the present disclosure are supplied by the gym it can be used as a complete marketing strategy for the gym, as there is no onus on the user to supply an item to facilitate usage of the system. This also allows product placements and adverting, as well as an e-commerce type.

It will be recognized that while certain aspects of the disclosure are described in terms of a specific sequence of steps of a method, these descriptions are only illustrative of the broader methods of the disclosure, and may be modified as required by the particular application. Certain steps may be rendered unnecessary or optional under certain circumstances. Additionally, certain steps or functionality may be added to the disclosed embodiments, or the order of performance of two or more steps permuted. All such variations are considered to be encompassed within the disclosure disclosed and claimed herein.

While the above detailed description has shown, described, and pointed out novel features of the disclosure as applied to various embodiments, it will be understood that various omissions, substitutions, and changes in the form and details of the device or process illustrated may be made by those skilled in the art without departing from the disclosure. The foregoing description is of the best mode presently contemplated of carrying out the disclosure. This description is in no way meant to be limiting, but rather should be taken as illustrative of the general principles of the disclosure. The scope of the disclosure should be determined with reference to the claims.

What is claimed is:

1. An interactive system for monitoring the physical activity of a user within a gym environment, comprising:
a local server having one or more databases storing personal data comprising at least an exercise history data and a pre-set exercise regime data for the user;
a smart device associated with a first gym equipment, the smart device having a display unit, a wireless communication apparatus and a processor, the smart device configured to permit an authenticated user to access and display their personal data on the display unit downloaded from the local server;
a wearable sensing device provided and assigned by the gym environment having a plurality of sensors configured to measure and record one or more exercise parameters of the user during use of the first gym equipment, the wearable sensing device further comprising a sensing device wireless communication apparatus;
the smart device being further configured to receive and process the recorded one or more exercise parameters from the wearable sensing device to thereby display and record real-time exercise statistics of the user during use of the first gym equipment on the display unit;
where the smart device is further configured to wirelessly transmit the exercise statistics to the local server to compile and update the exercise history data associated with the user.

2. The system of claim 1, where the system further comprises a remote database or a cloud storage configured to back-up the personal data via a wide area network (WAN).

3. The system of claim 2, where the user is permitted to access the personal data stored on the remote database or the cloud storage via a web portal following authentication therewith.

4. The system of claim 1, where the wearable sensing device is assigned a fixed IP address within a local area network (LAN) within the gym environment, the fixed IP address being assigned to the user upon logging into the system in order to assist the local server in recognizing the user during a workout period.

5. The system of claims 1, where the smart device is in communication with the wearable sensing device whereby the wearable sensing device is configured to permit the user to alter one or more exercise settings on the smart device.

6. The system of claim 5, where the one or more exercise settings comprises an amount of weight being lifted by the user during the exercise and/or a number of repetitions and sets being performed.

7. The system of claim 1, where the system further comprises a first near field communication (NFC) device associated with the smart device.

8. The system of claim 7, where the wearable sensing device comprises a second near field communication (NFC) device.

9. The system of claim 8, where bringing the first and second near field communication (NFC) devices in proximity to each other permits the user to be authenticated with the smart device.

10. The system of claim 1, where the real-time exercise statistics comprises one or more of intensity, rate of repetitions during the exercise, number of repetitions carried out during the exercise, heart-rate, and blood pressure.

11. The system of claim 1, where the wearable sensing device further comprises one or more magnetic sensors adapted to detect proximity with the first gym equipment.

12. The system of claim 1, where the wearable sensing device is configured to be attached to an item of clothing worn by the user.

13. The system of claim 1, where the wearable sensing device includes a wrist band configured to be secured around the wrist of the user.

14. The system of claim 1, where the local server transmits real-time information to the smart device to provide motivational indications to the user based on the recorded and processed one or more exercise parameters that are constantly compared to a pre-determined goal.

15. The system of claim 1, where the smart device is selected from the group consisting of a tablet device, a personal digital assistant (PDA) device and a computer.

16. A wearable sensing device, comprising:
a processor;
a power supply;
a motion sensor;
a storage apparatus; and
a wireless communication apparatus;
where the wearable sensing device is further configured to interact with an interactive system for monitoring the physical activity of a user within a gym environment.

17. The device of claim 16, where the motion sensor comprises:
at least three gravitational measurements;
at least three rotational measurements; and
at least three magnetic measurements.

18. The device of claim 17, where the motion sensor comprises one or more accelerometers.

19. The device of claim 16, where the device further comprises an input interface apparatus.

20. The device of claim 16, further comprising:
a status indicator light configured to provide the user with a visual indication of their performance during a particular exercise.
